(19) United States
(12) Patent Application Publication
(10) Pub. No.: US 2023/0374138 A1
(43) Pub. Date: Nov. 23, 2023

(54) ICOS TARGETING FOR NEUROPATHIC PAIN RELIEF

(71) Applicant: Board of Regents, The University of Texas System, Austin, TX (US)

(72) Inventors: Theodore J. Price, Dallas, TX (US); Ishwarya Sankaranarayanan, Allen, TX (US)

(73) Assignee: Board of Regents, The University of Texas System, Austin, TX (US)

(21) Appl. No.: 18/306,038

(22) Filed: Apr. 24, 2023

Related U.S. Application Data
(60) Provisional application No. 63/342,942, filed on May 17, 2022.

(51) Int. Cl. C07K 16/28; A61P 25/02; A61P 25/04

(52) U.S. Cl. CPC C07K 16/2818; A61P 25/02; A61P 25/04; C07K 2317/75

(57) ABSTRACT

Chemotherapy-induced peripheral neuropathy (CIPN) is a primary dose-limiting side effect caused by antineoplastic agents, such as paclitaxel. This causes damage to peripheral nerves and the dorsal root ganglia (DRG). Currently, there are no effective treatments for CIPN, which can lead to long-term morbidity in patients and survivors. Neuronal-immune interactions occur in CIPN and have been implicated both in the development and progression of the disease and disease resolution. The inventors investigated the potential role of Inducible co-stimulatory molecule (ICOS) in the resolution of CIPN. ICOS is an immune checkpoint molecule that is expressed on the surface of activated T cells and promotes proliferation and differentiation of T cells. They found that intrathecal administration of ICOS agonist antibody (ICOSaa) alleviates mechanical hypersensitivity caused by paclitaxel and facilitates the resolution of pain in female mice without a clear benefit in male mice. Administration of ICOSaa reduced astrocyte-gliosis in the spinal cord and satellite cell gliosis in the DRG of mice previously treated with paclitaxel. Mechanistically, ICOSaa treatment converted T cells in the DRG to an anti-inflammatory phenotype and also promoted pain resolution by increasing cytokine interleukin 10 (IL10) expression. In line with these observations, blocking IL10 activity occluded the effects of ICOSaa treatment on CIPN behavior in female mice. Suggesting a broader activity in neuropathic pain, ICOSaa also partially resolved mechanical hypersensitivity in the spared nerve injury (SNI) model. Our findings support a model wherein ICOSaa, upon engagement with T cells, induces an expansion of IL10 expression to facilitate neuropathic pain relief in female mice. ICOSaa treatment is in clinical development for solid tumors. Given our observation of resident T cells in the human DRG, ICOSaa therapy could be developed for testing in neuropathic pain clinical trials.

### PRIORITY CLAIM

This application claims benefit of priority to U.S. Provisional Application Ser. No. 63/342,942, filed May 17, 2022, the entire contents of which are hereby incorporated by reference.

### STATEMENT OF GOVERNMENT INTEREST

This invention was made with government support under Grant No. R01 NS065926 awarded by the National Institutes of Health. The government has certain rights in the invention.

### BACKGROUND INFORMATION

### 1. Field

The present disclosure relates generally to inducible co-stimulatory molecule (ICOS) for alleviating pain, and more specifically to ICOS targeting for alleviating Paclitaxel induced peripheral neuropathy via an IL-10-mediated mechanism in female mice.

### 2. Background

Chemotherapy-induced peripheral neuropathy (CIPN) is a debilitating condition due to the dose-limiting side effects of antineoplastic agents. Thirty-70% of patients receiving chemotherapy treatment experience numbness, reduced proprioception and pain. (Seretny et al., 2014). CIPN is predominately sensory-related, causing damage to the peripheral nervous system with some motor deficits (Boyette-Davis et al., 2013; Krishnan et al., 2005). Paclitaxel is a chemotherapy agent primarily used to treat breast, lung, and ovarian cancer. It acts through the stabilization of the microtubules which leads to mitotic arrest (Malacrida et al., 2019). Paclitaxel causes neurotoxicity by accumulating in the dorsal root ganglia (DRG) and in the peripheral nerves and impairing axonal transport and causing mitochondrial dysfunction that is linked to the neuropathy (Duggett et al., 2016; Flatters and Bennett, 2006; Gornstein and Schwarz, 2014; McWhinney et al., 2009; Megat et al., 2019; Park et al., 2013; Sahenk et al., 1994). Studies have shown that the immune system can play an active role in modulating chronic pain progression and resolution (Laumet et al., 2019b). Neuro-immune interactions occur in CIPN, and both the adaptive and innate immune systems play an essential role in the progression and resolution of neuropathic pain (Ji et al., 2016; Laumet et al., 2019b). For instance, T cells have been shown to secrete both pro- and anti-inflammatory cytokines such as IL6, IL5, IL4, IL10, IFNγ and TNFα that can interact with neurons and regulate chronic pain (Brandolini et al., 2019; Liu et al., 2014; Peters et al., 2007; Zhang et al., 2016).

T cell activation occurs when the T cell receptor (TCR) interacts with major histocompatibility complex I and II expressed on antigen-presenting cells (APC), dendritic cells (DC), macrophages, and B cells; however, secondary signaling is required for proper facilitation of T cell activation (Wikenheiser and Stumhofer, 2016). CD28 family of receptors provides this secondary signal for the activation and survival of T cells. Inducible co-stimulatory molecule (ICOS) is a member of the CD28 family, and an immune checkpoint receptor expressed on activated T cells (Amatore et al., 2018). It is known to induce an immune response by binding to its exclusive ICOS ligand (ICOSL) expressed on APC, DC, B cells, and tumor cells (Wikenheiser and Stumhofer, 2016). ICOS, upon activation, generates a signaling cascade on various subsets of T cells such as the CD4 T helper cells and CD8 cytotoxic T cells (Amatore et al., 2018, 2020). ICOS induction enhances proliferation and differentiation of T cells with the secretion of cytokines such as IL4, IL5, IL6, IL10, TNFα, and IL21 (Amatore et al., 2020; Maeda et al., 2011). ICOS-ICOSL signaling cascade plays a dual role, either by suppressing the activity on T regulatory cells or promoting proliferation and activation of T effector cells (Watanabe et al., 2008). This dual-purpose makes ICOS-ICOSL an attractive therapeutic target for cancer immunotherapy (Amatore et al., 2020).

Previous studies have demonstrated that cytokine signaling is an important driver of CIPN, and this is a primary mechanism of neuro-immune modulation (Brandolini et al., 2019; Laumet et al., 2019b; Megat et al., 2019). Paclitaxel promotes an increase in pro-inflammatory cytokines such as TNFα and IL1β with the suppression of anti-inflammatory cytokine IL10 and IL4 (Lees et al., 2017; Singh et al., 2022; Tonini et al., 2002). IL10, an anti-inflammatory cytokine, exerts a neuroprotective and pain-relieving effect in CIPN, osteoarthritis, and chronic constriction injury-induced neuropathic pain (Laumet et al., 2020; Milligan et al., 2006; Watkins et al., 2020). IL10 is secreted by innate immune cells like APC, DC, natural killer cells, macrophages and adaptive immune cells including Th1, Th2 and Treg subsets of T cells (Iyer and Cheng, 2012; Ng et al., 2013). The IL10 receptor (IL10R) is known to be expressed in the neurons of the DRG where its activation suppresses the excitability of nociceptors providing a plausible cellular mechanism for the relief of pain promoted by IL10 (Krukowski et al., 2016; Laumet et al., 2020).

Therefore, it would be desirable to have a therapy that take into account at least some of the issues discussed above, as well as other possible issues.

### SUMMARY

Thus, in accordance with the present disclosure, there is provided a method for treating pain in a subject having pain, the method comprising administering an agent to the subject that targets inducible co-stimulatory molecule (ICOS) signaling. The pain may be neuropathic pain, such as that caused by chemotherapy-induced peripheral neuropathy (CIPN) or by traumatic injury to the peripheral nervous system. The agent may be a protein or small molecule drug that operates to reduce astrocyte-gliosis in the spinal cord, reduce satellite cell gliosis in the root ganglia (DRG), convert T cells in the DRG to an anti-inflammatory phenotype, increase cytokine interleukin 10 (IL10) expression, or a combination thereof. The agent may be an inducible co-stimulatory molecule (ICOS) agonist antibody. Administering may include intrathecal administration. The pain in the subject may be reduced or eliminated. The pain in the subject may be caused by damage to peripheral nerves, the dorsal root ganglia (DRG), or a combination thereof, such as damage to peripheral nerves and/or the dorsal root ganglia (DRG) caused by one or more antineoplastic agents, such as paclitaxel.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C: Paclitaxel promotes infiltration of T cells into the DRG. FIG. 1A) Flow cytometry gating strategy for T cells in mice gated for CD45^{pos} singlets isolated from L3-L5 DRG on day 13 after paclitaxel treatment. FIG. 1B) Representative flow cytometry plots for CD3^{pos} T cells (previously gated for CD45^{pos} singlets) on day 13 in mice treated with paclitaxel or vehicle. FIG. 1C) Paclitaxel treatment was associated with a significant increase in the influx of T cells in the DRG measured by flow cytometry (unpaired t-test, t=3.601, p-value=0.0036, df=12) **p<0.01. N=7/group.

FIGS. 2A-E: ICOS agonist antibody (ICOSaa) promotes the resolution of paclitaxel-induced peripheral neuropathy. FIG. 2A) The cohorts of mice were subjected to intraperitoneal injection of 4 mg/kg paclitaxel or control every other day for a cumulative dosage of 16 mg/kg according to the schema shown followed by intrathecal injection of ICOSaa for four consecutive days. Arrows represent days of von Frey testing. Paclitaxel group represented in yellow and Ptx+ICOSaa represented in blue. FIG. 2B) Female mice reversed mechanical allodynia after intrathecal administration of ICOSaa (Two-way ANOVA, F=4.951, p-value<0.0001, post-hoc Sidak's, *Ptx+ICOSaa vs. Ptx, p-value=0.0318 at day 8, *Ptx+ICOSaa vs. Ptx, p-value=0.0323 at day 15, ***Ptx+ICOSaa vs. Ptx, p-value=0.0001 at day 28, **Ptx+ICOSaa vs. Ptx, p-value=0.0067 at day 32, *Ptx+ICOSaa vs. Ptx, p-value=0.0.0261 at day 36), N=8/group. FIG. 2C) Effect size was determined by calculating the cumulative difference between the value for each time point and the baseline value. The effect size difference was significant in the female cohort of mice (***Effect size, unpaired t test, t=4.963, p-value=0.0002, df=14). FIG. 2D) Male mice showed a trend in resolution of mechanical hypersensitivity measured with von Frey filaments after administration of ICOSaa but it was not significant (Two-way ANOVA, F=2.020, p-value=0.0544), N=6/group. FIG. 2E). The inventors did not observe any statistically significant differences between the groups in male (Effect size, unpaired t test, t=1.533, p-value=0.1564, df=10). *p<0.05, **p<0.01, ***p<0.001.

FIGS. 3A-C: ICOSaa reverses satellite cell gliosis in paclitaxel treated mice in the DRG. FIG. 3A) Representative images of GS (red) that labels satellite glial cells in the DRG, peripherin (blue). FIG. 3B) GS was reduced in animals treated with ICOSaa (One-way ANOVA, F=6.399, p-value=0.0128, post-hoc Tukey, *Vehicle vs. Ptx, p-value=0.0159, Ptx vs. Ptx+ICOSaa, p-value=0.0612) N=5/group. FIG. 3C) Nuclei were counted using Dapi staining; no significant difference was observed between the groups (One-way ANOVA, F=4.763, p-value=0.0577) N=3/group. Data are represented as mean±SEMs. *p<0.05 Scale bar=50 μm.

FIGS. 4A-D: ICOSaa reverses astrocyte gliosis in the spinal cord in paclitaxel treated mice. FIG. 4A) Representative image of astrocyte labeling using GFAP antibody in the spinal cord of Vehicle, paclitaxel, and paclitaxel+ICOSaa groups. FIG. 4B) Representative images of the dorsal horn of the spinal cord of vehicle, Ptx, and Ptx+ICOSaa animal cohorts stained with GFAP (green) for astrocyte labeling and Dapi (blue). FIG. 4C) Paclitaxel animals showed increased expression of GFAP in the dorsal horn of the spinal cord. This was significantly reduced in mice treated with ICOSaa (One-way ANOVA, F=18.42, p-value=0.0002, post-hoc Tukey, ***Vehicle vs. Ptx, p-value=0.0002, **Ptx vs. Ptx+ICOSaa, p-value=0.0048, Vehicle vs. Ptx+ICOSaa, p-value=0.2485) N=5/group. FIG. 4D) No significant change was observed in the number of nuclei using Dapi (One-way ANOVA, F=4.409, p-value=0.0367, post-hoc Tukey, *Vehicle vs. Ptx, p-value=0.0360, Ptx vs. Ptx+ICOSaa, p-value=0.3365, Vehicle vs. Ptx+ICOSaa, p-value=0.7129) N=5/group. Data are represented as mean±SEMs. *p<0.05, **p<0.01, ***p<0.001 Scale bar=100 μm and 200 μm respectively.

FIGS. 5A-H: IL-10R antagonist reverses the effect of ICOSaa treatment in mice treated with paclitaxel. FIG. 5A) Schematic representation of experimental design. Arrows represent days with von Frey testing. Groups are shown as: Vehicle+IL10Ra (Grey), Ptx+IgG (Yellow), Ptx+ICOSaa+IgG (Blue), Ptx+IL10Ra (Green) and Ptx+ICOSaa+IL10Ra (Pink). FIG. 5B) Mechanical nociceptive thresholds are shown for each group with statistical differences represented in the graph (Two-way ANOVA, F=1.921, p-value=0.0048, post-hoc Bonferroni, *Vehicle+IL10Ra vs. Ptx+IgG, p-value=0.0362, Vehicle+IL10Ra vs. Ptx+ICOSaa+IgG, p-value=ns, *Vehicle+IL10Ra vs. Ptx+IL10Ra, p-value=0.0485, *Vehicle+IL10Ra vs. Ptx+ICOSaa+IL10Ra, p-value=0.0452, ^{$}Ptx+IgG vs Ptx+ICOSaa+IgG, p-value=0.0574, Ptx+IgG vs Ptx+IL10Ra, p-value=ns, Ptx+IgG vs. Ptx+ICOSaa+IL10Ra, p-value=ns, ^{#}Ptx+ICOSaa+IgG vs. Ptx+IL10Ra, p-value=0.0697, *Ptx+ICOS+IgG vs. Ptx+ICOS+IL10Ra, p-value=0.0697, Ptx+ICOSaa+IL10Ra vs. Ptx+IL10Ra, p-value=ns). N=4, 5, 5, 5, 5 per group, respectively. FIG. 5C) The effect size was significant between Ptx+ICOSaa+IL10Ra and Ptx+ICOSaa+IgG1 (One-way ANOVA, F=7.211, p-value=0.0010, post-hoc Tukey, *Vehicle+IL10Ra vs. Ptx+IgG, p-value=0.0108, Vehicle+IL10Ra vs. Ptx+ICOSaa+IgG, p-value=ns, *Vehicle+IL10Ra vs. Ptx+IL10Ra p-value=0.0173, **Vehicle+IL10Ra vs. Ptx+ICOSaa+IL10Ra, p-value=0.0079, *Ptx+IgG vs. Ptx+ICOSaa+Ig G, p-value=0.0301, Ptx+IgG vs. Ptx+IL10Ra, p-value=ns, *Ptx+ICOSaa+IgG vs. Ptx+IL10Ra, p-value=0.0485, Ptx+IL10Ra+IgG vs. Ptx+ICOSaa+IL10Ra, p-value=ns). FIG. 5D) A significant increase in IL10 expression was observed in mice subjected to intrathecal injection of ICOSaa using ELISA (One-way ANOVA, F=9.452, p-value=0.0061, post-hoc Tukey, Vehicle vs. Ptx, p-value=ns, **Vehicle vs. Ptx+ICOSaa, p-value=0.0069, *Ptx vs. Ptx+ICOSaa, p-value=0.0216) N=4/group. FIG. 5E) Representative flow cytometry plots of subset of T cells CD4 positive and CD8 positive (previously gated on CD45^{pos}CD3^{pos}) in each group after treatment, isolated on day 13 from L3-L5 DRG. FIG. 5F) Frequency of CD3 positive T cells was increased in Ptx and Ptx+ICOSaa groups compared to vehicle and no significant changes were observed between Ptx and Ptx+ICOSaa (One-way ANOVA, F=6.052, p-value=0.0118, post-hoc Tukey, *Vehicle vs. Ptx, p-value=0.02, *Vehicle vs. Ptx+ICOSaa, p-value=0.0357, Ptx vs. Ptx+ICOSaa, p-value=ns) N=6/group. FIG. 5G) Percentage of CD4 positive T cells was increased in Ptx and Ptx+ICOSaa groups compared to vehicle treated cohort and no significant changes were observed between Ptx and Ptx+ICOSaa groups (One-way ANOVA, F=10.86, p-value=0.004, post-hoc Tukey, **Vehicle vs. Ptx, p-value=0.0073, **Vehicle vs. Ptx+ICOSaa, p-value=0.076, Ptx vs. Ptx+ICOSaa, p-value=ns) N=4/group. FIG. 5H) Percentage of CD8 positive T cells were increased only in Ptx+ICOSaa group compared to vehicle control (One-way ANOVA, F=4.881, p-value=0.0367, post-hoc Tukey, Vehicle vs. Ptx, p-value=ns, *Vehicle vs. Ptx+ICOSaa, p-value=0.0301, Ptx vs. Ptx+ICOSaa p-value=ns), N=4/group, *p<0.05, **p<0.01.

FIGS. 6A-C: ICOSaa partially reverses mechanical allodynia in female SNI mice. FIG. 6A) Schematic representation of groups subjected to SNI on day one followed by intrathecal injection of ICOSaa on days 14-17. Arrows represent days with von Frey testing. FIG. 6B) Mechanical hypersensitivity was partially reversed only on days 17 and 19 in the group administered with ICOSaa group (Two-way ANOVA, F=3.473, p-value<0.0001, post-hoc Bonferroni, ***SNI vs. SNI+ICOSaa at day 17, p-value=0.0006, ***SNI vs. SNI+ICOSaa at day 19, p-value=0.0003), N=7/group. FIG. 6C) Effect size was determined by calculating the cumulative difference between the value for each time point and the baseline value. The effect size was significant between SNI and SNI+ICOSaa treated mice (One-way ANOVA, F=18.0, p-value<0.0001, post-hoc Tukey, *Sham vs. SNI+ICOSaa, p-value=0.0265, ***Sham vs. SNI, p=value<0.0001, *SNI vs. SNI+ICOS, p-value=0.0151). *p<0.05, * ***p<0.001.

FIGS. 7A-B: T cell expression in human DRG. FIG. 7A) representative image of CD4 (white) peripherin (purple), Dapi (blue) T cell expression in human dorsal root ganglion. FIG. 7B) representative image of CD8 (white) peripherin (purple), Dapi (blue) T cell expression in human DRG. The white arrows point towards CD4 or CD8 T cell. Scale bar=50 μm and 20 μm, respectively.

### DETAILED DESCRIPTION

The illustrative embodiments recognize and take into account one or more different considerations. Based on this foundation of evidence, the inventors hypothesized that activation of ICOS signaling on T cells in the DRG, or in the vicinity of the DRG such as the spinal meninges (Da Mesquita et al., 2021; Louveau et al., 2018; Salvador et al., 2021), could lead to the secretion of IL10 cytokine and alleviate paclitaxel-induced peripheral neuropathy.

The primary goal was to determine if ICOSaa treatment could promote pain resolution in paclitaxel-induced peripheral neuropathy. The inventors found that paclitaxel leads to T cell infiltration in the DRG, which supports the idea of targeting ICOS signaling for pain resolution. Administration of ICOSaa attenuated hind paw hypersensitivity in female mice previously treated with paclitaxel. ICOSaa also reduced astrogliosis in the dorsal horn of the spinal cord and satellite cell gliosis in the DRG. ICOSaa treatment led to enhanced expression of anti-inflammatory cytokine IL10 in the DRG in T cells. Consistent with this, IL10 blocking treatment occluded the beneficial effect of ICOSaa treatment in the CIPN model. These findings demonstrate a new mechanism for stimulating T cells to promote pain resolution via ICOSaa treatment.

### I. INDUCIBLE T-CELL COSTIMULATOR

Inducible T-cell costimulator (ICOS), also known as CD278, is an immune checkpoint protein that in humans is encoded by the ICOS gene. ICOS is a CD28-superfamily costimulatory molecule that is expressed on activated T cells. It is thought to be important for Th2 cells in particular. The protein encoded by this gene belongs to the CD28 and CTLA-4 cell-surface receptor family. It forms homodimers and plays an important role in cell-cell signaling, immune responses and regulation of cell proliferation.

Compared to wild-type naïve T cells, ICOS^{-/-} T cells activated with plate-bound anti-CD3 have reduced proliferation and IL-2 secretion. The defect in proliferation can be rescued by addition of IL-2 to the culture, suggesting the proliferative defect is due either to ICOS-mediated IL-2 secretion or the activation of similar signaling pathways between ICOS and IL-2. In terms of Th1 and Th2 cytokine secretion, ICOS^{-/-} CD4+ T cell activated in vitro reduced IL-4 secretion, while maintaining similar IFN-γ secretion. Similarly, CD4+ T cells purified from ICOS^{-/-} mice immunized with the protein keyhole limpet hemocyanin (KLH) in alum or complete Freund's Adjuvant have attenuated IL-4 secretion, but similar IFN-g and IL-5 secretion when recalled with KLH.

These data are similar to an airway hypersensitivity model showing similar IL-5 secretion, but reduced IL-4 secretion in response to sensitization with Ova protein, indicating a defect in Th2 cytokine secretion, but not a defect in Th1 differentiation as both IL-4 and IL-5 are Th2-associated cytokines. In agreement with reduced Th2 responses, ICOS^{-/-} mice expressed reduced germinal center formation and IgG1 and IgE antibody titers in response to immunization.

### II. PAIN

Pain is an unpleasant feeling often caused by intense or damaging stimuli. The International Association for the Study of Pain's widely used definition states: “Pain is an unpleasant sensory and emotional experience associated with actual or potential tissue damage or described in terms of such damage.”

Pain motivates the individual to withdraw from damaging situations, to protect a damaged body part while it heals, and to avoid similar experiences in the future. Most pain resolves promptly once the painful stimulus is removed and the body has healed, but sometimes pain persists despite removal of the stimulus and apparent healing of the body; and sometimes pain arises in the absence of any detectable stimulus, damage or disease.

Pain is the most common reason for physician consultation in the United States. It is a major symptom in many medical conditions and can significantly interfere with a person's quality of life and general functioning. Psychological factors such as social support, hypnotic suggestion, excitement, or distraction can significantly modulate pain's intensity or unpleasantness.

The International Association for the Study of Pain (IASP) has classified pain according to specific characteristics: (a) region of the body involved (e.g., abdomen, lower limbs), (b) system whose dysfunction may be causing the pain (e.g., nervous, gastrointestinal), (c) duration and pattern of occurrence, (d) intensity and time since onset, and (e) etiology. This system has been criticized by Clifford J. Woolf and others as inadequate for guiding research and treatment. According to Woolf, there are three classes of pain: nociceptive pain (see hereunder), inflammatory pain which is associated with tissue damage and the infiltration of immune cells, and pathological pain which is a disease state caused by damage to the nervous system (neuropathic pain, see hereunder) or by its abnormal function (dysfunctional pain, like in fibromyalgia, irritable bowel syndrome, tension type headache, etc.).

A. Chronic Pain

Pain is usually transitory, lasting only until the noxious stimulus is removed or the underlying damage or pathology has healed, but some painful conditions, such as rheumatoid arthritis, peripheral neuropathy, cancer and idiopathic pain, may persist for years. Pain that lasts a long time is called chronic, and pain that resolves quickly is called acute. Traditionally, the distinction between acute and chronic pain has relied upon an arbitrary interval of time from onset; the two most commonly used markers being 3 months and 6 months since the onset of pain, though some theorists and researchers have placed the transition from acute to chronic pain at 12 months. Others apply acute to pain that lasts less than 30 days, chronic to pain of more than six months duration, and subacute to pain that lasts from one to six months. A popular alternative definition of chronic pain, involving no arbitrarily fixed durations is “pain that extends beyond the expected period of healing.” Chronic pain may be classified as cancer pain or benign.

B. Nociceptive Pain

Nociceptive pain is caused by stimulation of peripheral nerve fibers that respond only to stimuli approaching or exceeding harmful intensity (nociceptors) and may be classified according to the mode of noxious stimulation; the most common categories being “thermal” (heat or cold), “mechanical” (crushing, tearing, etc.) and “chemical” (iodine in a cut, chili powder in the eyes). As subset of nociceptive pain is called “inflammatory” pain, as it results from tissue damage and the response of innate inflammatory responses. Nociceptive pain may also be divided into “visceral,” “deep somatic” and “superficial somatic” pain. Visceral structures are highly sensitive to stretch, ischemia and inflammation, but relatively insensitive to other stimuli that normally evoke pain in other structures, such as burning and cutting. Visceral pain is diffuse, difficult to locate and often referred to a distant, usually superficial, structure. It may be accompanied by nausea and vomiting and may be described as sickening, deep, squeezing, and dull. Deep somatic pain is initiated by stimulation of nociceptors in ligaments, tendons, bones, blood vessels, fasciae and muscles, and is dull, aching, poorly localized pain. Examples include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns.

C. Neuropathic Pain

Neuropathic pain is pain caused by damage or disease that affects the somatosensory system. It may be associated with abnormal sensations called dysesthesia, and pain produced by normally non-painful stimuli (allodynia). Neuropathic pain may have continuous and/or episodic (paroxysmal) components. The latter are likened to an electric shock. Common qualities include burning or coldness, “pins and needles” sensations, numbness and itching. Nociceptive pain, by contrast, is more commonly described as aching.

Neuropathic pain may result from disorders of the peripheral nervous system or the central nervous system (brain and spinal cord). Thus, neuropathic pain may be divided into peripheral neuropathic pain, central neuropathic pain, or mixed (peripheral and central) neuropathic pain. Central neuropathic pain is found in spinal cord injury, multiple sclerosis, and some strokes. Aside from diabetes (see diabetic neuropathy) and other metabolic conditions, the common causes of painful peripheral neuropathies are herpes zoster infection, HIV-related neuropathies, nutritional deficiencies, toxins, remote manifestations of malignancies, immune mediated disorders and physical trauma to a nerve trunk.

Neuropathic pain is common in cancer as a direct result of cancer on peripheral nerves (e.g., compression by a tumor), or as a side effect of chemotherapy, radiation injury or surgery. After a peripheral nerve lesion, aberrant regeneration may occur. Neurons become unusually sensitive and develop spontaneous pathological activity, abnormal excitability, and heightened sensitivity to chemical, thermal and mechanical stimuli. This phenomenon is called “peripheral sensitization.”

### IV. ANTIBODIES

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A. General Methods

In accordance with the present disclosure, it will be understood that monoclonal antibodies binding to ICOS will have applications in the treatment of pain. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (mAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce ICOS-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies with the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the mAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EB V-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

mAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### V. PHARMACEUTICAL FORMULATIONS AND METHODS OF TREATING PAIN

Treating pain and avoiding tolerance to pain killers are major issues in clinical medicine. One goal of current research is to find ways to improve the efficacy of pain relief, as well as prevent the development of tolerance or addiction, and reduce side effects. One way is by combining such traditional therapies with the therapies of the present invention. In the context of the present invention, it is contemplated that an ICOS antagonist may be used, optionally in a combination therapy with an opiate for chronic use.

The present disclosure provides pharmaceutical compositions comprising anti-ICOS antibodies. Such compositions comprise a prophylactically or therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Passive transfer of antibodies generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

For combination therapies, the agents would be provided in a combined amount effective to reduce tolerance and to reduce side effects associated with the opioid, including but not limited to addiction and withdrawal. This process may involve contacting the patient with the agents/therapies at the same time. This may be achieved by contacting the patient with a single composition or pharmacological formulation that includes both agents, or by contacting the cell with two distinct compositions or formulations, at the same time, wherein one composition includes the A₃AR agonist and the other includes the opiate.

Alternatively, the treatment according to the present invention may precede or follow the other treatment by intervals ranging from minutes to weeks. In embodiments where the opiate and the A₃AR agonist are applied separately to the subject, one would generally ensure that a significant period of time did not expire between each delivery, such that the therapies would still be able to exert an advantageously combined effect on the subject. In such instances, it is contemplated that one would administer both modalities within about 12-24 hours of each other, within about 6-12 hours of each other, or with a delay time of only about 12 hours. In some situations, it may be desirable to extend the time period for treatment significantly; however, several days (2, 3, 4, 5, 6 or 7) to several weeks (1, 2, 3, 4, 5, 6, 7 or 8) may lapse between the respective administrations.

It also is conceivable that more than one administration of either the A₃AR agonist or the opioid therapy will be desired. Various combinations may be employed, where the A₃AR agonist is “A,” and the opioid therapy is “B,” as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B
   Other combinations, including chronic and continuous dosing of one or both agents, are contemplated.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Animals. ICR mice were maintained and bred at the animal facility at the University of Texas at Dallas. Experiments were performed using 8-12 weeks old female and male littermates. The mice were housed (4 maximum/cage) with food and water ad libitum in a 12 h light-dark cycle and maintained at room temperature (21±2° C.). All procedures were approved by the Institutional Animal Care and Use Committee at University of Texas at Dallas.

Injections. Paclitaxel was prepared in 50% El Kolipher (Sigma-Aldrich) and 50% ethanol. Mice received 4 mg/kg of paclitaxel every other day, for a cumulative intraperitoneal dosage of 16 mg/kg, or vehicle control (50% El Kolipher and 50% ethanol diluted in PBS). Intrathecal injections of Inducible co-stimulatory (ICOS) agonist antibody (C398.4 Biolegend, 0.5 μg/μl) in 5 μl volume were done using a 30.5-gauge needle and Hamilton syringe for four consecutive days under isoflurane anesthesia. For the IL10 blocking treatment, mice received 250 μg of InVivoMAb anti-mouse IL10R antibody (BioXcell CD210) or InVivoMAb rat IgG1 isotype control, anti-horseradish peroxidase antibody (BioX cell) vehicle control twice weekly until the end of the experiment.

Mechanical Withdrawal Threshold. Mice were habituated one hour before testing for mechanical hypersensitivity in a clear acrylic behavioral chamber. Mechanical paw withdrawal threshold was tested using the up-down method (Chaplan et al., 1994) using calibrated von Frey filaments (Stoelting) perpendicular to the mid plantar surface of the hind paw. A positive response consisted of an immediate flicking or licking behavior upon applying the filament to the hind paw. The investigator was blinded during all days of testing.

Flowcytometry Analysis. Mice were euthanized under isoflurane anesthesia by cervical dislocation on day 13 after intrathecal injection of ICOSaa. L3-L5 DRGs were dissected and minced using scissors in the lysis buffer containing 1.6 mg/mL collagenase (Worthington), 10 mM HEPES (Thermo Fisher Scientific), and 5 mg/ml of Bovine serum albumin (Thermo Fisher Scientific) at 37° C. for thirty minutes. The digested tissue was passed through a 70 μm cell strainer and the samples were centrifuged at 600×g at 4° C. for 5 min. The supernatant was discarded and the cell pellet were incubated with red blood cell (RBC) lysis buffer (Biolegend) for 5 mins at room temperature followed by a wash with 1% Bovine serum albumin in Phosphate buffer saline (PBS). The cell pellet was stained for anti-mouse CD3-PE (1:200, 145-2C11 Biolegend), anti-mouse CD45-APC/Cy7 (1:200, 30-F11 Biolegend), anti-mouse CD8a-PE/Cy7 (53-6.7 Biolegend), anti-mouse CD4-PE-Cyanine 5.5 (RM4-5 Biolegend) for 30 mins in the dark at 4° C. followed by three washes with 1% bovine serum albumin in PBS (Thermo Fisher Scientific). The cell samples were acquired using BD LSR Fortessa (BD Biosciences) flow cytometer using BDFACS DIVA software (BD Biosciences) and analyzed with FlowJo software (v.10, FlowJo). Flow cytometry analysis was performed by first gating for CD45 positive hematopoietic cells followed by gating for single cell lymphocytes. All T cells were identified as CD45^{pos}+CD3^{pos}, subset of T helper cells as CD45^{pos}+CD3^{pos}+CD4^{pos} and cytotoxic T cell subset as CD45^{pos}+CD3^{pos}+CD8a^{pos}.

Immunohistochemistry. Mice were euthanized under isoflurane anesthesia by cervical dislocation on day 13 after intrathecal injection of ICOSaa. DRGs and spinal cord were dissected and frozen in optimum cutting temperature (OCT) medium (Fisher Scientific). 20 μm sections were cut using the cryostat and placed on the charged side of the slides. The tissue sections were fixed in 4% cold formaldehyde (Thermo Fisher Scientific) for 10 minutes, followed by dehydration with increasing ethanol concentrations from 50%, 75%, and 100% for 5 minutes each. The sections were blocked for one hour using 10% normal goat serum (R &D systems) and 0.3% Triton-X (Sigma-Aldrich). The tissue sections were incubated with primary antibodies GFAP, Glutamine Synthetase, DAPI, Peripherin, CD8a, CD4 (Table 1) diluted in blocking solution at 4° C. for overnight, followed by the corresponding secondary antibody diluted in blocking solution for one hour at room temperature. The slides were washed in 0.1M PB and cover-slipped using Prolong Gold Antifade (Fisher Scientific P36930). Images were taken on Olympus FluoView 1200 confocal microscope and Olympus FluoView 3000 confocal microscope, using the same settings for all images.

Image analysis of the dorsal horn of the lumbar spinal cord were obtained by calculating the corrected total cell fluorescence (CTCF) intensity of GFAP and Dapi using the formula CTCF=Integrated Density−(Area of selected cell×Mean fluorescence of background readings). CTCF values were normalized by the area the images captured and analyses of spinal cord images were done using ImageJ version 1.48 (National Institutes of Health, Bethesda, MD). A total of 3 sections per animal was analyzed.

L3-L5 DRG images were taken using the Olympus FluoView 3000 confocal microscope using the same setting for all images. A region of interest (ROI) was drawn around individual neurons to measure the mean grey intensity value (MGI). Background fluorescence was also measured using negative control subjected to blocking and secondary antibody with no primary. An average intensity was calculated after subtracting the MGI values of negative control and normalized over the area of the individual ROI. Nuclei were individually counted around each ROI and normalized by area. Image analysis was performed using Olympus cellSens software.

TABLE 1
List of Antibodies Used

| Antibodies | Company | Catalog # | Dilution |
|---|---|---|---|
| Glutamine Synthetase | Thermo | Cat # 11307- | 1:1000 |
| polyclonal antibody | fisher | 2-AP | |
| Peripherin | EnCor | Cat # CPCA- | 1:1000 |
| | Biotechnology | Peri | |
| | Inc | | |
| Dapi | Cayman | Item no, 14285 | 1:5000 |
| Anti-GFAP | Neuro Mab | N206A/8 - | 1:1000 |
| | | 75-240 | |
| IgG (H + L) Cross- | Fisher | A21428 | 1:2000 |
| Adsorbed Goat anti- | Scientific | | |
| Rabbit, Alexa Fluor ® 555 | | | |
| IgY (H + L) Goat anti- | Fisher | A21449 | 1:2000 |
| Chicken, Alexa Fluor ® 647 | Scientific | | |

Enzyme-linked immunosorbent Assay (ELISA). DRGs were dissected on day 13 after the last administration of ICOSaa, and flash frozen on dry ice. The frozen tissue was homogenized using a sonicator in lysis buffer (50 mM Tris, pH 7.4, 150 mM NaCl, one mM EDTA, pH 8.0, and 1% Triton X-100) containing protease and phosphatase inhibitors (Sigma-Aldrich) and briefly centrifuged to extract the protein from the supernatant. Pierce BCA Protein Assay (Thermo Fisher Scientific) was performed by following the manufacturer's protocol to identify the amount of protein in each sample. Enzyme-linked immunosorbent assay to detect IL10 (Thermo Fisher Scientific—88-7105-22) cytokine was performed according to the manufacturer's instructions.

Surgery. Spare nerve injury was performed as previously described (Decosterd and Woolf, 2000), leaving the sural branch and cutting the peroneal and tibial branches at the left sciatic nerve trifurcation. Sham control was performed the same way but without cutting any nerve. Mice were allowed to recover for two weeks post-surgery before administration of the ICOSaa treatment and testing for mechanical von Frey thresholds.

Data analysis and Statistics. All analyses and data were generated using GraphPad Prism 8.4.1. Statistical analysis between groups were determined using one or two-way ANOVA, followed by Bonferroni, Sidak or Tukey post hoc tests. Differences between two groups were assessed using the Student's t-test. Statistical results can be found in the figure legends. Effect sizes were determined by subtracting behavior scores for each time point from baseline measures. Absolute values were summed up and plotted for each group. All data were represented as mean+/−SEM with p<0.05 considered significant. The sample size and sex are noted in the graphs and figure legends.

### Example 2—Results

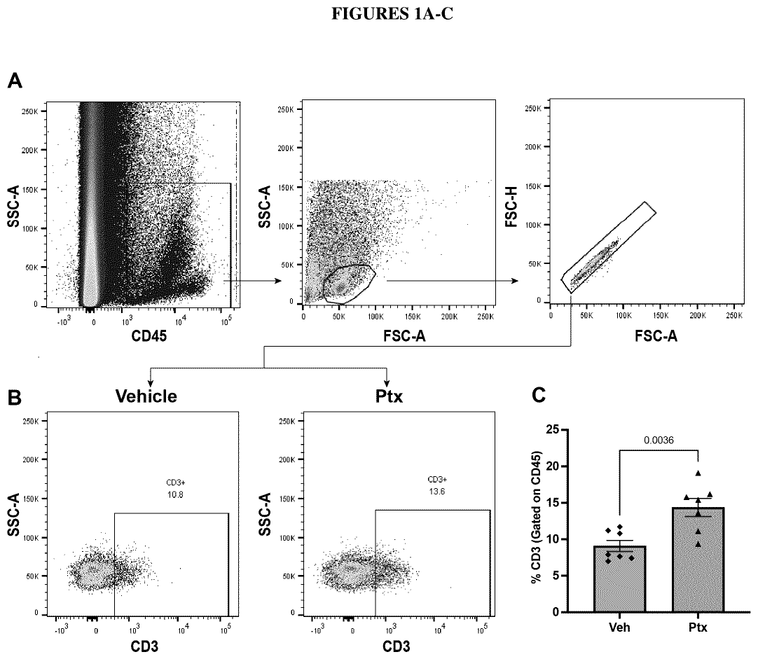

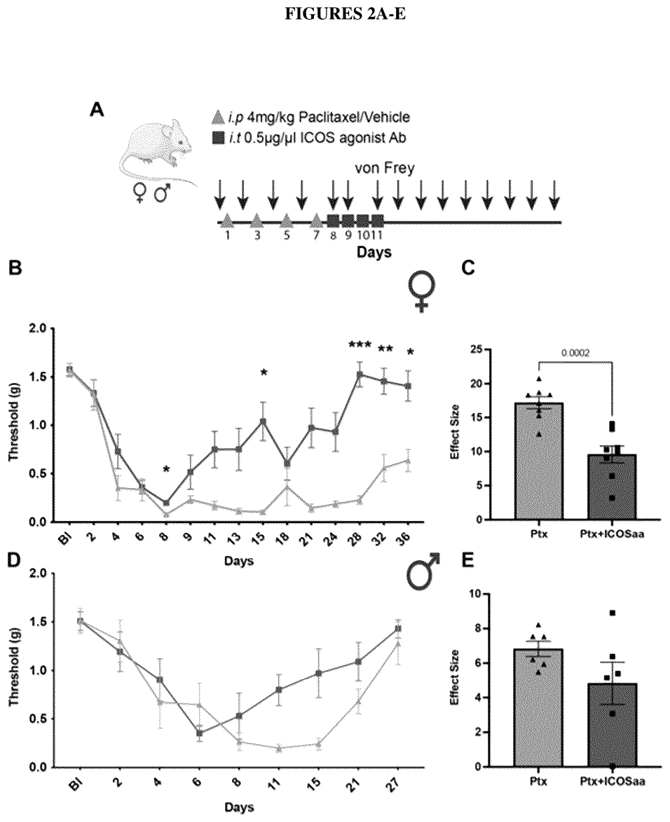

ICOSaa accelerates the resolution of paclitaxel-induced peripheral neuropathy in female mice. T cells activation can occur with the engagement of ICOS similar to CD28 (Lohning et al., 2003; Mahajan et al., 2007). For that reason, the inventors first assessed the immune response after paclitaxel administration using flow cytometry. They measured the number of CD3 positive T cells by gating for live singlets with CD45 positive lymphocytes, followed by subsequent gating for CD3 positive cells (FIGS. 1A-B). They observed a significant increase in the percentage of CD3 positive T cells in the DRG on day 10 after the start of the treatment in mice that received paclitaxel compared to vehicle control (FIG. 1C). Having confirmed an increase in the number of T cells in the DRG in mice treated with paclitaxel, the inventors then tested whether the ICOSaa would have any effect on paclitaxel-induced mechanical hypersensitivity. To do this, the treated both male and female mice with paclitaxel every other day for a total of 4 injections and cumulative dose of 16 mg/kg, which produced mechanical hypersensitivity in both sexes (FIG. 2A). On the final day of paclitaxel treatment, the inventors started once daily treatment of ICOSaa (0.5 μg/μL) or vehicle given intrathecally for 4 consecutive days. Qualitatively, animals of both sexes treated with ICOSaa showed a more rapid resolution of mechanical hypersensitivity, but a significant treatment effect was only seen in female mice by two-way anova (FIGS. 2B and 2D). There was also a significant effect on the overall effect size of ICOSaa treatment in female but not male mice (FIGS. 2C and E). Given this sex difference in the effect of ICOSaa, and the far greater proportion of women treated with paclitaxel for cancer, the inventors focused on the female mice for the remainder of the study.

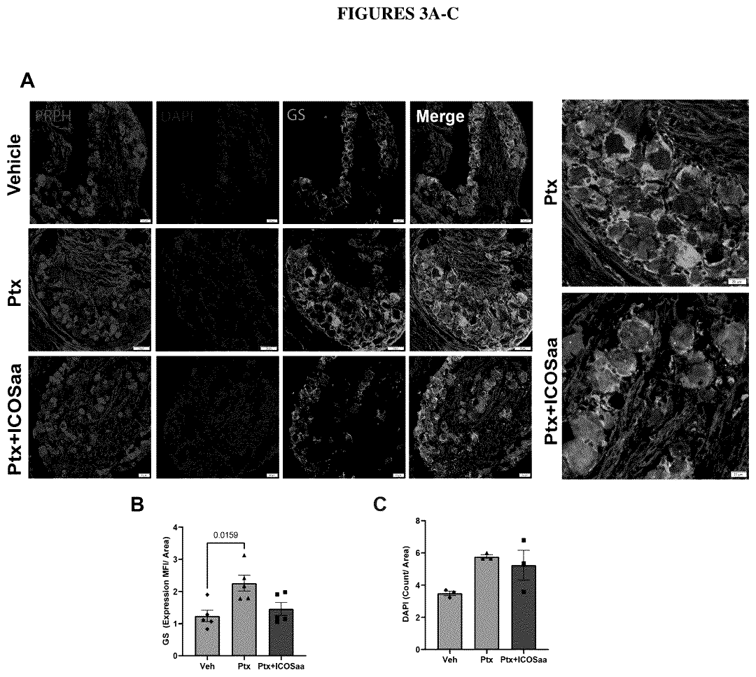

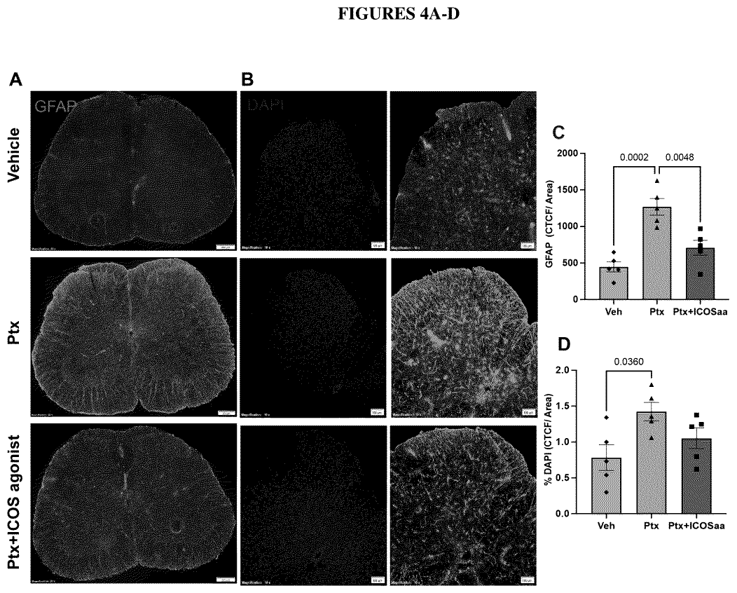

ICOSaa reverses satellite cell gliosis in the DRG and astrogliosis in the spinal cord in paclitaxel treated female mice. Since the inventors observed a resolution of mechanical allodynia in the ICOS-treated animals, they next examined glial cell changes in the DRG and the spinal cord (Leo et al., 2021; Makker et al., 2017; Robinson el al., 2014). Gliosis is a sign of injury or stress to the tissue, where the cells become hyperactive and change morphology (Pekny and Pekna, 2014). The inventors performed immunohistochemistry to quantify the expression of glutamine synthetase (GS) in satellite glial cells (SGC) in the DRG and GFAP in astrocytes in the dorsal horn of lumbar spinal cord. They observed an increase in GS expression in paclitaxel treated animals and a reduction in animals treated with ICOSaa (FIGS. 3A-B). No significant changes were observed in the number of Dapi positive nuclei between the groups (FIGS. 3A and 3C). This suggests that administration of paclitaxel alone or with ICOSaa does not cause SOC proliferation and, instead, reversed their hyperactive state. Expression of GFAP protein was increased in the dorsal horn of lumbar spinal cord in paclitaxel-treated animals and this was reduced in cohorts subjected to ICOSaa treatment (FIGS. 4A-C). In addition, there was no substantial change in the number of Dapi positive nuclei between the ICOS treated and paclitaxel treated animals (FIGS. 4B and 4D), suggesting that glial cells were not proliferating.

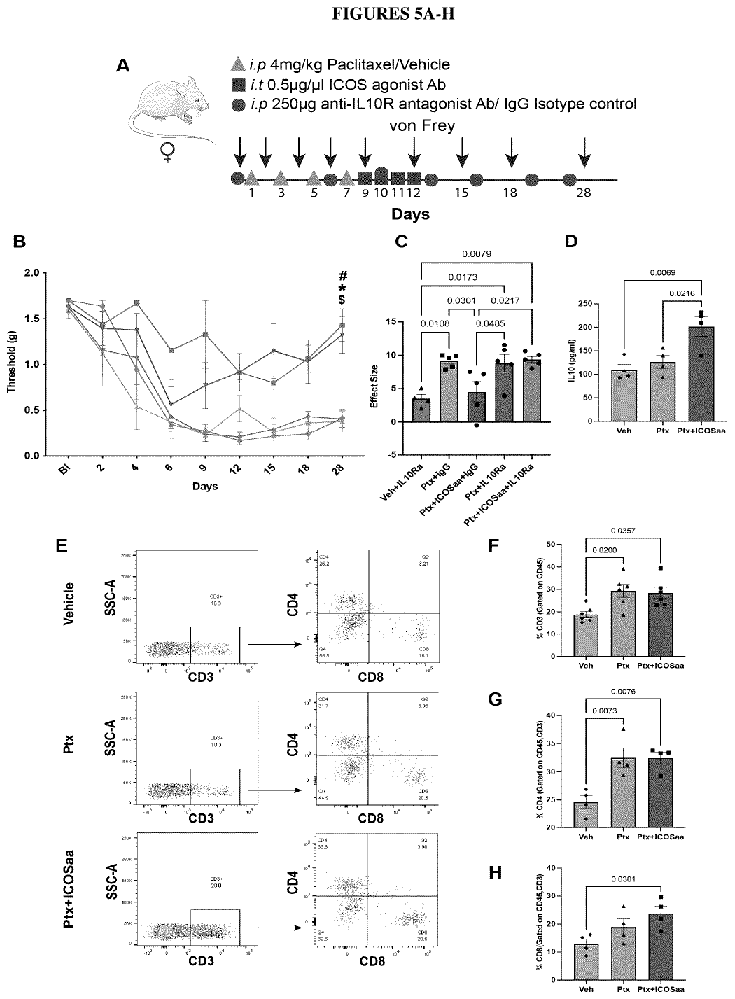

IL-10R antagonist blocks paclitaxel-induced mechanical hypersensitivity in mice treated with ICOSaa. Paclitaxel treated female mice that were then treated with ICOSaa showed a significant reduction in mechanical hypersensitivity. The inventors hypothesized that this effect could be driven by IL10 based on multiple studies demonstrating the T cell-derived IL10 can resolve neuropathic pain, in particular in female mice (Krukowski et al., 2016; Laumet et al., 2020; Ledeboer et al., 2007). They injected IL10R antagonist antibody or isotype control twice every week to block the activity of IL10 at IL10R, using the same paclitaxel and ICOSaa dosing schedule as described above (FIG. 5A). They observed that while the IL10R antagonist antibody had no effect on its own, it completely blocked the effect of ICOSaa treatment in paclitaxel treated female mice (FIG. 5B). This finding suggests that the mechanism by which ICOSaa resolves paclitaxel-induced mechanical hypersensitivity is through the secretion of anti-inflammatory IL10. Next, the inventors assessed if IL10 expression could be increased in the DRG after ICOSaa administration. They measured IL10 concentration in the DRG on day 13, the day after the end of ICOSaa treatment, using an ELISA assay. They observed a significant increase in production of IL10 cytokine in the ICOSaa treated cohort compared to paclitaxel or vehicle control (FIG. 5C).

Building on the inventors' observation that paclitaxel administration recruits T cells into the DRG (FIG. 1), they sought to further examine the subsets of T cells infiltrating the DRG after administration of ICOSaa. They performed flow cytometry analysis by gating for T cell subsets as CD45^{pos}CD3^{pos}CD4^{pos} and CD45^{pos}CD3^{pos}CD8a^{pos} (FIG. 5E). Paclitaxel induced infiltration of CD45^{pos}CD3^{pos}, T cells, but no significant changes were observed for this cell type compared to the ICOSaa treated group (FIG. 5F). The inventors saw no significant differences in the percentage of CD4 and CD8 positive T cells between ICOSaa treated cohort with the paclitaxel treated mice (FIGS. 5G and 5H). They observed an increase in the frequency of CD8 positive T cells in ICOSaa mice. This cell type is known to promote upregulation of IL10 in the DRG—(Krukowski et al., 2016; Laumet et al., 2019a). This further suggests that ICOSaa activates the T cells to secrete endogenous IL10 promoting the resolution of paclitaxel induced neuropathy.

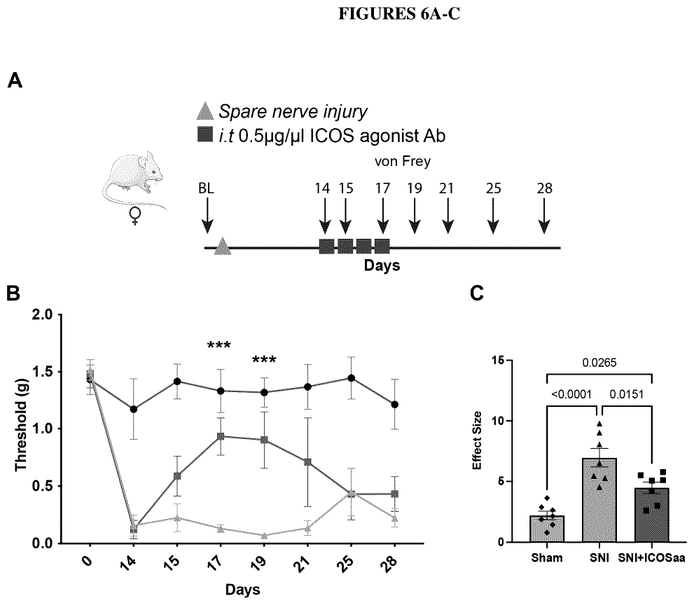

ICOS agonist alleviates mechanical hypersensitivity in the SNI model. The inventors next investigated if ICOSaa treatment could have an effect on mechanical hypersensitivity in the SNI neuropathic pain model. They performed SNI or sham surgery and administered four consecutive doses of ICOSaa two weeks post-surgery (FIG. 6A). They observed an inhibition of mechanical hypersensitivity with ICOSaa treatment in female SNI mice on days 17 and 19 post SNI. This effect was transient and lasted only until the last dose of ICOS administration (FIG. 6B). The effect size was also significant in animals treated ICOSaa compared to vehicle (FIG. 6C).

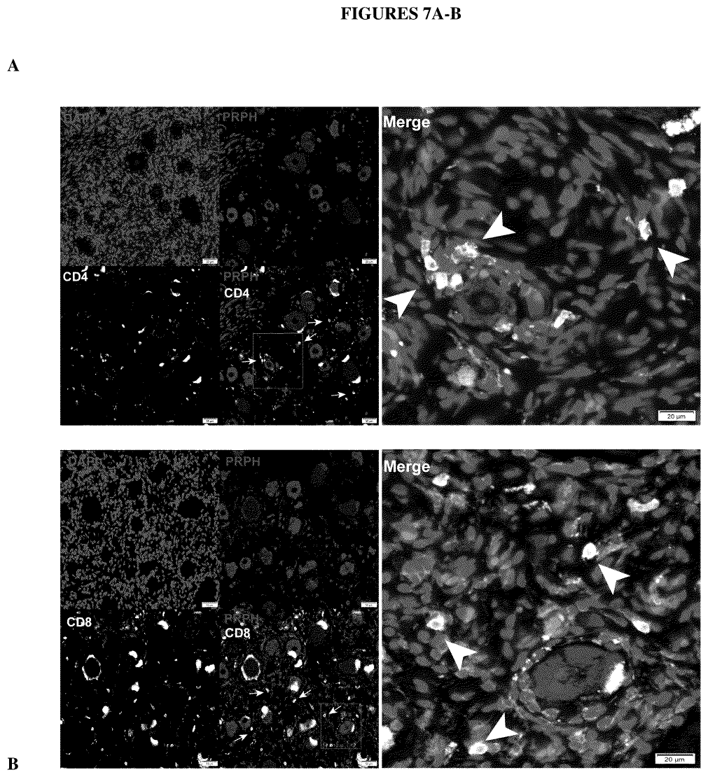

Presence of T cells in human DRG. These findings suggest that T cells in the DRG can be manipulated by ICOSaa treatment to increase IL10 expression and alleviate neuropathic pain. To examine the translational potential of this approach, the inventors investigated whether T cells are found in the human DRG using DRGs recovered from organ donors. They performed IHC using markers for CD4 and CD8a T cells. They observed that both CD4 and CD8a T cells were present in the DRG of organ donors, with many of these cells clustered around neurons (FIG. 7). This is contrast with findings in naïve mouse DRG, where T cells are not present or are found only in low numbers (Krukowski et al., 2016). This finding supports the translational potential of ICOSaa treatment for neuropathic pain.

### Example 3—Discussion

In this study, the inventors demonstrate that targeting ICOS molecule on T cells facilitates the resolution of neuropathic pain in female mice. The engagement of ICOS on the surface of activated T cells induces the production of anti-inflammatory cytokine IL10, that subsequently led to reversal of mechanical allodynia. The inventors' findings are in line with literature that T cells can play a beneficial role in promoting pain resolution (Krukowski et al., 2016; Laumet et al., 2019b; Mao et al., 2017). This study reveals a completely new strategy for exogenous modulation of T cells to facilitate pain resolution. This work shows that T cells infiltrate into the DRG after paclitaxel treatment. This is likely critical for pain resolution induced by ICOSaa as ICOS is only present on the surface of activated T cells (Austin et al., 2012; Lees et al., 2015). In a recent study, CD8+ T cells were shown to alleviate CIPN only in the presence of cisplatin via an increase in the expression of IL10R in the DRG (Krukowski et al., 2016; Laumet et al., 2019a). These observations are consistent with the inventors' results that administration of paclitaxel promotes T cells infiltration into the DRG and ICOSaa directs these cells towards a pain resolution phenotype.

ICOSaa treatment has been shown to mitigate disease severity in autoimmune disorders, lupus and tumors (Amatore et al., 2018). ICOS-ICOSL signaling has dual functions. On the one hand, it can suppress T regulatory cells and on the other it can cause T effector cells to express and secrete anti-inflammatory cytokines such as IL4 and IL10 (Watanabe et al., 2008). Here, the inventors show that administration of ICOSaa induces T cells into activating ICOS-ICOSL signaling cascade to release anti-inflammatory cytokine IL10. ICOSaa treatment led to resolution of mechanical hypersensitivity in the paclitaxel treated female mice and also reversed satellite cell gliosis in the DRG and astocyte gliosis in the dorsal horn of the spinal cord. This action on cellular measures of CIPN suggests the possibility of disease-modifying properties of ICOSaa treatment. Further studies will be needed to ascertain if ICOSaa treatment can also prevent the development of CIPN with paclitaxel treatment and/or if it is effective in other species. The observation of T cells in the DRGs of organ donors is promising from the perspective of clinical translation for these findings.

Cytokines are an important group of molecules that regulate the excitability of nociceptors and for this reason are intensively investigated for the treatment of pain where agonist and antagonist cytokine therapies are under development (Inyang et al., 2021). IL10 has been shown to alleviate CIPN and rheumatoid arthritis (Watkins et al., 2020). In vitro studies show that ICOSaa activates the secretion of anti-inflammatory cytokines such as IL4 and IL10 (Arimura et al., 2002). These results are in line with this literature where the inventors have shown that ICOSaa regulates T cells in the DRG to secrete the anti-inflammatory cytokine IL10. IL10Ra is expressed on sensory neurons in the rodent DRG, and also on human DRG neurons (Tavares-Ferreira et al., 2022), and its activation in rodent nociceptors reduces their excitability (Laumet et al., 2020). Although IL10 therapy can alleviate neuropathic pain, IL10 has a short half-life under physiological conditions and the plasmid-based DNA therapy which showed promising results on canines with osteoarthritis is still not FDA approved (Soderquist et al., 2010; Watkins et al., 2020). The inventors' model of educating T cells to release IL10 with ICOSaa may be able to overcome some of these limitations with a therapeutic approach that is nearing the clinic for other indications (Solinas et al., 2020).

The inventors observed a transient anti-nociceptive effect with the administration of ICOSaa in the SNI model of neuropathic pain. A possible explanation for this result could be that the mechanisms for the development and maintenance of pain in the SNI model is not entirely T cell dependent. The role of T cells in trauma-induced neuropathic pain is still somewhat controversial. A previous study using the SNI model in Rag1−/− mice (T cell deficient mice) reported less mechanical allodynia (Costigan et al., 2009). However, another study using the same model showed that T cells likely play a more important role in promoting neuropathic pain in female mice than in male mice (Sorge et al., 2015). Since this time, it has become clear that the type of T cell is critical for understanding the impact of these cells on neuropathic pain and neuropathic pain resolution. Again, the strategy of targeting ICOS to increase IL10 expression in CD8 T cells is a new approach that could have clinical translation potential.

A key finding of this study is the presence of CD8+ T cells in human DRG collected from healthy donors. This strengthens the case for the translational potential of ICOSaa. In naïve mice there are very few T cells present in the DRG but the efficacy of ICOSaa treatment is facilitated by the paclitaxel challenge promoting the infiltration of T cells into the DRG (Krukowski et al., 2016). This work demonstrates that both CD4+ and CD8+ T cells are present in human, healthy DRGs and are in the vicinity of neurons. To date, multiple Phase I and II clinical trials are being conducted for ICOS agonists and ICOS antagonists due to the high expression of ICOS in the tumor microenvironment and duality of function for the treatment of advance solid tumors (Garber, 2020; Le Tourneau et al., 2020). The inventors believe that their findings support the possibility that ICOS agonists could be used for pain treatment.

### VII. REFERENCES

The following references, to the extent that they provide exemplary procedural or other details supplementary to those set forth herein, are specifically incorporated herein by reference.
Amatore, F., Gorvel, L., Olive, D., 2018. Inducible Co-Stimulator (ICOS) as a potential therapeutic target for anti-cancer therapy. Expert Opin Ther Targets 22, 343-351.
Amatore, F., Gorvel, L., Olive, D., 2020. Role of Inducible Co-Stimulator (ICOS) in cancer immunotherapy. Expert Opin Biol Ther 20, 141-150.
Arimura, Y., Kato, H., Dianzani, U., Okamoto, T., Kamekura, S., Buonfiglio, D., Miyoshi-Akiyama, T., Uchiyama, T., Yagi, J., 2002. A co-stimulatory molecule on activated T cells, H4/ICOS, delivers specific signals in T(h) cells and regulates their responses. Int Immunol 14, 555-566.
Austin, P. J., Kim, C. F., Perera, C. J., Moalem-Taylor, G., 2012. Regulatory T cells attenuate neuropathic pain following peripheral nerve injury and experimental autoimmune neuritis. Pain 153, 1916-1931.
Boyette-Davis, J. A., Cata, J. P., Driver, L. C., Novy, D. M., Bruel, B. M., Mooring, D. L., Wendelschafer-Crabb, G., Kennedy, W. R., Dougherty, P. M., 2013. Persistent chemoneuropathy in patients receiving the plant alkaloids paclitaxel and vincristine. Cancer Chemother Pharmacol 71, 619-626.
Brandolini, L., d'Angelo, M., Antonosante, A., Allegretti, M., Cimini, A., 2019. Chemokine Signaling in Chemotherapy-Induced Neuropathic Pain. Int J Mol Sci 20.
Chaplan, S. R., Bach, F. W., Pogrel, J. W., Chung, J. M., Yaksh, T. L., 1994. Quantitative assessment of tactile allodynia in the rat paw. J Neurosci Methods 53, 55-63.
Costigan, M., Moss, A., Latremoliere, A., Johnston, C., Verma-Gandhu, M., Herbert, T. A., Barrett, L., Brenner, G. J., Vardeh, D., Woolf, C. J., Fitzgerald, M., 2009. T-cell infiltration and signaling in the adult dorsal spinal cord is a major contributor to neuropathic pain-like hypersensitivity. J Neurosci 29, 14415-14422.
Da Mesquita, S., Papadopoulos, Z., Dykstra, T., Brase, L., Farias, F. G., Wall, M., Jiang, H., Kodira, C. D., de Lima, K. A., Herz, J., Louveau, A., Goldman, D. H., Salvador, A. F., Onengut-Gumuscu, S., Farber, E., Dabhi, N., Kennedy, T., Milam, M. G., Baker, W., Smirnov, I., Rich, S. S., Dominantly Inherited Alzheimer, N., Benitez, B. A., Karch, C. M., Perrin, R. J., Farlow, M., Chhatwal, J. P., Holtzman, D. M., Cruchaga, C., Harari, O., Kipnis, J., 2021. Meningeal lymphatics affect microglia responses and anti-Abeta immunotherapy. Nature 593, 255-260.
Decosterd, I., Woolf, C. J., 2000. Spared nerve injury: an animal model of persistent peripheral neuropathic pain. Pain 87, 149-158.
Duggett, N. A., Griffiths, L. A., McKenna, O. E., de Santis, V., Yongsanguanchai, N., Mokori, E. B., Flatters, S. J., 2016. Oxidative stress in the development, maintenance and resolution of paclitaxel-induced painful neuropathy. Neuroscience 333, 13-26.
Flatters, S. J., Bennett, G. J., 2006. Studies of peripheral sensory nerves in paclitaxel-induced painful peripheral neuropathy: evidence for mitochondrial dysfunction. Pain 122, 245-257.
Garber, K., 2020. Immune agonist antibodies face critical test. Nature reviews. Drug discovery 19, 3-5.
Gornstein, E., Schwarz, T. L., 2014. The paradox of paclitaxel neurotoxicity: Mechanisms and unanswered questions. Neuropharmacology 76 Pt A, 175-183.
Inyang, K. E., Folger, J. K., Laumet, G., 2021. Can FDA-Approved Immunomodulatory Drugs be Repurposed/Repositioned to Alleviate Chronic Pain? J Neuroimmune Pharmacol 16, 531-547.
Iyer, S. S., Cheng, G., 2012. Role of interleukin 10 transcriptional regulation in inflammation and autoimmune disease. Crit Rev Immunol 32, 23-63.
Ji, R. R., Chamessian, A., Zhang, Y. Q., 2016. Pain regulation by non-neuronal cells and inflammation. Science 354, 572-577.
Krishnan, A. V., Goldstein, D., Friedlander, M., Kiernan, M. C., 2005. Oxaliplatin-induced neurotoxicity and the development of neuropathy. Muscle Nerve 32, 51-60.
Krukowski, K., Eijkelkamp, N., Laumet, G., Hack, C. E., Li, Y., Dougherty, P. M., Heijnen, C. J., Kavelaars, A., 2016. CD8+ T Cells and Endogenous IL-10 Are Required for Resolution of Chemotherapy-Induced Neuropathic Pain. J Neurosci 36, 11074-11083.
Laumet, G., Bavencoffe, A., Edralin, J. D., Huo, X. J., Walters, E. T., Dantzer, R., Heijnen, C. J., Kavelaars, A., 2020. Interleukin-10 resolves pain hypersensitivity induced by cisplatin by reversing sensory neuron hyperexcitability. Pain 161, 2344-2352.
Laumet, G., Edralin, J. D., Dantzer, R., Heijnen, C. J., Kavelaars, A., 2019a. Cisplatin educates CD8+ T cells to prevent and resolve chemotherapy-induced peripheral neuropathy in mice. Pain 160, 1459-1468.
Laumet, G., Ma, J., Robison, A. J., Kumari, S., Heijnen, C. J., Kavelaars, A., 2019b. T Cells as an Emerging Target for Chronic Pain Therapy. Front Mol Neurosci 12, 216.
Le Tourneau, C., Rischin, D., Groenland, S., Lim, A., Martin-Liberal, J., Moreno, V., Trigo, J.,
Mathew, M., Cho, D., Hansen, A., 2020. 1O Inducible T cell co-stimulatory (ICOS) receptor agonist, GSK3359609 (GSK609) alone and combination with pembrolizumab: Preliminary results from INDUCE-1 expansion cohorts in head and neck squamous cell carcinoma (HNSCC). Annals of Oncology 31, Si.
Ledeboer, A., Jekich, B. M., Sloane, E. M., Mahoney, J. H., Langer, S. J., Milligan, E. D., Martin, D., Maier, S. F., Johnson, K. W., Leinwand, L. A., Chavez, R. A., Watkins, L. R., 2007. Intrathecal interleukin-10 gene therapy attenuates paclitaxel-induced mechanical allodynia and proinflammatory cytokine expression in dorsal root ganglia in rats. Brain Behav Immun 21, 686-698.
Lees, J. G., Duffy, S. S., Perera, C. J., Moalem-Taylor, G., 2015. Depletion of Foxp3+ regulatory T cells increases severity of mechanical allodynia and significantly alters systemic cytokine levels following peripheral nerve injury. Cytokine 71, 207-214.
Lees, J. G., Makker, P. G., Tonkin, R. S., Abdulla, M., Park, S. B., Goldstein, D., Moalem-Taylor, G., 2017. Immune-mediated processes implicated in chemotherapy-induced peripheral neuropathy. Eur J Cancer 73, 22-29.
Leo, M., Schmitt, L.-I., Kutritz, A., Kleinschnitz, C., Hagenacker, T., 2021. Cisplatin-induced activation and functional modulation of satellite glial cells lead to cytokine-mediated modulation of sensory neuron excitability. Experimental Neurology 341, 113695.
Liu, X. J., Zhang, Y., Liu, T., Xu, Z. Z., Park, C. K., Berta, T., Jiang, D., Ji, R. R., 2014. Nociceptive neurons regulate innate and adaptive immunity and neuropathic pain through MyD88 adapter. Cell Res 24, 1374-1377.
Lohning, M., Hutloff, A., Kallinich, T., Mages, H. W., Bonhagen, K., Radbruch, A., Hamelmann, E., Kroczek, R. A., 2003. Expression of ICOS in vivo defines CD4+ effector T cells with high inflammatory potential and a strong bias for secretion of interleukin 10. The Journal of experimental medicine 197, 181-193.
Louveau, A., Herz, J., Alme, M. N., Salvador, A. F., Dong, M. Q., Viar, K. E., Herod, S. G., Knopp, J., Setliff, J. C., Lupi, A. L., Da Mesquita, S., Frost, E. L., Gaultier, A., Harris, T. H., Cao, R., Hu, S., Lukens, J. R., Smirnov, I., Overall, C. C., Oliver, G., Kipnis, J., 2018. CNS lymphatic drainage and neuroinflammation are regulated by meningeal lymphatic vasculature. Nat Neurosci 21, 1380-1391.
Maeda, S., Fujimoto, M., Matsushita, T., Hamaguchi, Y., Takehara, K., Hasegawa, M., 2011. Inducible costimulator (ICOS) and ICOS ligand signaling has pivotal roles in skin wound healing via cytokine production. The American journal of pathology 179, 2360-2369.
Mahajan, S., Cervera, A., MacLeod, M., Fillatreau, S., Perona-Wright, G., Meek, S., Smith, A., MacDonald, A., Gray, D., 2007. The role of ICOS in the development of CD4 T cell help and the reactivation of memory T cells. European journal of immunology 37, 1796-1808.
Makker, P. G., Duffy, S. S., Lees, J. G., Perera, C. J., Tonkin, R. S., Butovsky, O., Park, S. B., Goldstein, D., Moalem-Taylor, G., 2017. Characterisation of immune and neuroinflammatory changes associated with chemotherapy-induced peripheral neuropathy. PloS one 12, e0170814.
Malacrida, A., Meregalli, C., Rodriguez-Menendez, V., Nicolini, G., 2019. Chemotherapy-induced peripheral neuropathy and changes in cytoskeleton. International journal of molecular sciences 20, 2287.
Mao, L., Li, P., Zhu, W., Cai, W., Liu, Z., Wang, Y., Luo, W., Stetler, R. A., Leak, R. K., Yu, W., Gao, Y., Chen, J., Chen, G., Hu, X., 2017. Regulatory T cells ameliorate tissue plasminogen activator-induced brain haemorrhage after stroke. Brain 140, 1914-1931.
McWhinney, S. R., Goldberg, R. M., McLeod, H. L., 2009. Platinum neurotoxicity pharmacogenetics. Mol Cancer Ther 8, 10-16.
Megat, S., Ray, P. R., Moy, J. K., Lou, T. F., Barragan-Iglesias, P., Li, Y., Pradhan, G., Wanghzou, A., Ahmad, A., Burton, M. D., North, R. Y., Dougherty, P. M., Khoutorsky, A., Sonenberg, N., Webster, K. R., Dussor, G., Campbell, Z. T., Price, T. J., 2019. Nociceptor Translational Profiling Reveals the Ragulator-Rag GTPase Complex as a Critical Generator of Neuropathic Pain. J Neurosci 39, 393-411.
Milligan, E. D., Sloane, E. M., Langer, S. J., Hughes, T. S., Jekich, B. M., Frank, M. G., Mahoney, J. H., Levkoff, L. H., Maier, S. F., Cruz, P. E., 2006. Repeated intrathecal injections of plasmid DNA encoding interleukin-10 produce prolonged reversal of neuropathic pain. Pain 126, 294-308.
Ng, T. H., Britton, G. J., Hill, E. V., Verhagen, J., Burton, B. R., Wraith, D. C., 2013. Regulation of adaptive immunity; the role of interleukin-10. Front Immunol 4, 129.
Park, S. B., Goldstein, D., Krishnan, A. V., Lin, C. S., Friedlander, M. L., Cassidy, J., Koltzenburg, M., Kiernan, M. C., 2013. Chemotherapy-induced peripheral neurotoxicity: a critical analysis. CA Cancer J Clin 63, 419-437.
Pekny, M., Pekna, M., 2014. Astrocyte reactivity and reactive astrogliosis: costs and benefits. Physiological reviews 94, 1077-1098.
Peters, C. M., Jimenez-Andrade, J. M., Jonas, B. M., Sevcik, M. A., Koewler, N. J., Ghilardi, J. R., Wong, G. Y., Mantyh, P. W., 2007. Intravenous paclitaxel administration in the rat induces a peripheral sensory neuropathy characterized by macrophage infiltration and injury to sensory neurons and their supporting cells. Exp Neurol 203, 42-54.
Robinson, C. R., Zhang, H., Dougherty, P. M., 2014. Astrocytes, but not microglia, are activated in oxaliplatin and bortezomib-induced peripheral neuropathy in the rat. Neuroscience 274, 308-317.
Sahenk, Z., Barohn, R., New, P., Mendell, J. R., 1994. Taxol neuropathy. Electrodiagnostic and sural nerve biopsy findings. Arch Neurol 51, 726-729.
Salvador, A. F., de Lima, K. A., Kipnis, J., 2021. Neuromodulation by the immune system: a focus on cytokines. Nat Rev Immunol 21, 526-541.
Seretny, M., Currie, G. L., Sena, E. S., Ramnarine, S., Grant, R., MacLeod, M. R., Colvin, L. A., Fallon, M., 2014. Incidence, prevalence, and predictors of chemotherapy-induced peripheral neuropathy: A systematic review and meta-analysis. Pain 155, 2461-2470.
Singh, A. K., Mahalingam, R., Squillace, S., Jacobson, K. A., Tosh, D. K., Dharmaraj, S., Farr, S. A., Kavelaars, A., Salvemini, D., Heijnen, C. J., 2022. Targeting the A₃ adenosine receptor to prevent and reverse chemotherapy-induced neurotoxicities in mice. Acta Neuropathol Commun 10, 11.
Soderquist, R. G., Milligan, E. D., Harrison, J. A., Chavez, R. A., Johnson, K. W., Watkins, L. R., Mahoney, M. J., 2010. PEGylation of interleukin-10 for the mitigation of enhanced pain states. J Biomed Mater Res A 93, 1169-1179.
Solinas, C., Gu-Trantien, C., Willard-Gallo, K., 2020. The rationale behind targeting the ICOS-ICOS ligand costimulatory pathway in cancer immunotherapy. ESMO Open 5.
Sorge, R. E., Mapplebeck, J. C., Rosen, S., Beggs, S., Taves, S., Alexander, J. K., Martin, L. J., Austin, J. S., Sotocinal, S. G., Chen, D., Yang, M., Shi, X. Q., Huang, H., Pillon, N. J., Bilan, P. J., Tu, Y., Klip, A., Ji, R. R., Zhang, J., Salter, M. W., Mogil, J. S., 2015. Different immune cells mediate mechanical pain hypersensitivity in male and female mice. Nat Neurosci 18, 1081-1083.
Tavares-Ferreira, D., Shiers, S., Ray, P. R., Wangzhou, A., Jeevakumar, V., Sankaranarayanan, I., Cervantes, A. M., Reese, J. C., Chamessian, A., Copits, B. A., Dougherty, P. M., Gereau, R. W.t., Burton, M. D., Dussor, G., Price, T. J., 2022. Spatial transcriptomics of dorsal root ganglia identifies molecular signatures of human nociceptors. Sci Transl Med 14, eabj8186.
Tonini, G., Santini, D., Vincenzi, B., Borzomati, D., Dicuonzo, G., La Cesa, A., Onori, N., Coppola, R., 2002. Oxaliplatin may induce cytokine-release syndrome in colorectal cancer patients. Journal of biological regulators and homeostatic agents 16, 105-109.
Watanabe, M., Takagi, Y., Kotani, M., Hara, Y., Inamine, A., Hayashi, K., Ogawa, S., Takeda, K., Tanabe, K., Abe, R., 2008. Down-regulation of ICOS ligand by interaction with ICOS functions as a regulatory mechanism for immune responses. J Immunol 180, 5222-5234.
Watkins, L. R., Chavez, R. A., Landry, R., Fry, M., Green-Fulgham, S. M., Coulson, J. D., Collins, S. D., Glover, D. K., Rieger, J., Forsayeth, J. R., 2020. Targeted interleukin-10 plasmid DNA therapy in the treatment of osteoarthritis: Toxicology and pain efficacy assessments. Brain Behav Immun 90, 155-166.
Wikenheiser, D. J., Stumhofer, J. S., 2016. ICOS Co-Stimulation: Friend or Foe? Front Immunol 7, 304.
Zhang, H., Li, Y., de Carvalho-Barbosa, M., Kavelaars, A., Heijnen, C. J., Albrecht, P. J., Dougherty, P. M., 2016. Dorsal Root Ganglion Infiltration by Macrophages Contributes to Paclitaxel Chemotherapy-Induced Peripheral Neuropathy. J Pain 17, 775-786.

1. A method for treating pain in a subject having pain, the method comprising:
administering an agent to the subject that targets inducible co-stimulatory molecule (ICOS) signaling.

2. The method of claim 1, wherein the pain is neuropathic pain.

3. The method of claim 1, wherein the agent is a protein or small molecule drug that operates to reduce astrocyte-gliosis in the spinal cord, reduce satellite cell gliosis in the root ganglia (DRG), convert T cells in the DRG to an anti-inflammatory phenotype, increase cytokine interleukin 10 (IL10) expression, or a combination thereof.

4. The method of claim 1, wherein the pain in the subject is caused by chemotherapy-induced peripheral neuropathy (CIPN) or by traumatic injury to the peripheral nervous system.

5. The method of claim 1, wherein the agent is an inducible co-stimulatory molecule (ICOS) agonist antibody.

6. The method of claim 1, wherein administering includes intrathecal administration.

7. The method of claim 1, wherein the pain in the subject is reduced or eliminated.

8. The method of claim 1, wherein the pain in the subject is caused by damage to peripheral nerves, the dorsal root ganglia (DRG), or a combination thereof.

9. The method of claim 8, wherein the damage to peripheral nerves and/or the dorsal root ganglia (DRG) is caused by one or more antineoplastic agents.

10. The method of claim 9, wherein the one or more antineoplastic agents comprises paclitaxel.
